(19) United States
(12) Patent Application Publication
(10) Pub. No.: US 2020/0254046 A1
(43) Pub. Date: Aug. 13, 2020

(54) PHARMACEUTICAL COMPOSITIONS

(71) Applicant: ASTRAZENECA AB, Södertälje (SE)

(72) Inventors: Christian VON CORSWANT, Mölndal (SE); Romain BORDES, Göteborg (SE); Simon Peter Michael HJELM JONASSON, Göteborg (SE)

(21) Appl. No.: 16/316,228

(22) PCT Filed: Jul. 6, 2017
(86) PCT No.: PCT/EP2017/066983

§ 371 Date: Jan. 8, 2019

(30) Foreign Application Priority Data
Jul. 8, 2016 (GB) 1611920.8

(51) Int. Cl. A61K 36/48; A61K 9/16; A61K 31/201; A61K 31/202; A61K 9/20

(52) U.S. Cl. CPC A61K 36/48; A61K 9/1652; A61K 9/1611; A61K 9/20; A61K 31/202; A61K 9/1623; A61K 31/201

(57) ABSTRACT

The present application relates to solid pharmaceutical compositions and solid dosage forms containing them which comprise oils as their active pharmaceutical ingredient. Methods of preparing the compositions and their uses as described.

The present application relates to solid pharmaceutical compositions and solid dosage forms containing them which comprise oils as their active pharmaceutical ingredient. Methods of preparing the compositions and their uses are described.

The options for preparation of pharmaceutical formulations of active ingredients which are oils at room temperature (for example between 15° C. and 35° C.) are severely limited. For example, pharmaceutical compositions rich in polyunsaturated fatty acids (PUFAs), such as omega-3 PUFAs, which are being developed for a variety of clinical indications are often presented as oil filled gelatin capsules. These capsules can be significant in size as the dosages can be large (for example up to four 1 g capsules for treatment of hypertriglyceridemia). Omega-3 PUFAs may also conveniently be prepared as part of combination products, particularly for treatment of cardiovascular conditions where patients may require a number of different medicines. However presentation of the PUFAs in gelatin capsules limits the number of approaches available for formulating fixed dose combinations.

It would be convenient to prepare tablet dosage forms containing active ingredients which are oils, such as omega-3 PUFAs, either as sole active ingredient, in combination with other pharmaceutical active agents or possibly acting as a carrier for another active ingredient.

An emulsion is a system of two immiscible liquids where one of the liquids has been dispersed in the other by addition of an emulsifier. Emulsions may be either oil-in-water (o/w) emulsions where the oil is dispersed in a continuous phase of water, or conversely water-in-oil emulsions where oil is the continuous phase. The emulsifier is generally a surface active molecule, but particles can also be used as emulsifiers to produce stable emulsions. Pickering oil-in-water emulsions are distinguished from other oil-in-water emulsions by the presence of solid particles at the oil-water interface. Pickering emulsions where the solid particles are comprised of cellulose nanocrystals bound with a water-soluble polymer such as a cellulose derivative (for example hydroxypropyl methyl cellulose (HPMC)) have been described.

Cranston et al (ACS Sustainable Chem Eng, 2015, 3, 1023-1031) described synergistic stabilisation of emulsions and emulsion gels with water soluble polymers and cellulose nanocrystals (CNCs). Cranston et al (ACS Macro Lett, 2016, 5, 185-189) described dried and re-dispersible cellulose nanocrystal pickering emulsions containing tannic acid. Surprisingly we have found that pickering emulsions of a fatty acid oil phase stabilised by CNCs and a water soluble polymer such as one or more polymeric cellulose derivatives can be spray-dried into a stable powder, wherein the CNCs and cellulose derivative(s) form a solid matrix in which the oil remain dispersed. The resulting powder may be encapsulated or provided in a sachet or as a granulate, but surprisingly, the resulting powder can alternatively be compressed into tablet dosage forms without significant escape or loss of the oil during the compression process. This provides the potential to provide patient-friendly tablets of oils, such as omega-3 PUFAs, as well as to create fixed dose combinations with other active ingredients by admixing the combination prior to compression, or by spray coating the compressed oil-containing tablets with a coating containing a second active ingredient. Advantageously, dispersion of the spray dried powder in water reforms the emulsion with droplets similar to their original size, indicating that the spray drying process has not caused significant change to the system. Furthermore, re-dispersion of the tableted powders also does not appear to cause significant change to the system.

Therefore in a first aspect there is provided a solid pharmaceutical composition comprising

i) a powder comprising an active pharmaceutical ingredient which exists as an oil at least between 15° C. and 35° C., dispersed in a solid matrix, said solid matrix comprising cellulose nanocrystals and at least one cellulose derivative; and

ii) one or more pharmaceutically-acceptable excipients.

In another aspect there is provided a solid pharmaceutical composition comprising

i) a powder comprising an active pharmaceutical ingredient which exists as an oil at least between 15° C. and 35° C., dispersed in a solid matrix, said solid matrix comprising cellulose nanocrystals, at least one cellulose derivative and a pharmaceutically-acceptable salt of a polyvalent metal cation; and

ii) one or more pharmaceutically-acceptable excipients.

In another aspect there is provided a solid pharmaceutical composition comprising

i) a powder formed by spray-drying an emulsion, said emulsion comprising at least one cellulose derivative, water, cellulose nanocrystals, a pharmaceutically-acceptable salt of a polyvalent metal cation and an active pharmaceutical ingredient which exists as an oil at least between 15° C. and 35° C.; and

ii) one or more pharmaceutically-acceptable excipients.

In a further aspect there is provided a solid pharmaceutical composition comprising

i) a powder formed by spray-drying an emulsion which is formed by steps a to e:
a) dissolving at least one cellulose derivative in water;
b) dispersing cellulose nanocrystals in the resulting solution;
c) optionally adding a pharmaceutically-acceptable salt of a polyvalent metal cation;
d) adding an active pharmaceutical ingredient which is an oil between at least 15° C. and 35° C.; and
e) emulsifying the resulting mixture; and

ii) one or more pharmaceutically-acceptable excipients.

Suitably the pharmaceutically-acceptable salt of a polyvalent metal cation is a soluble pharmaceutically-acceptable calcium salt, such as calcium chloride.

Suitably the cellulose derivative is selected from HPMC (hydroxypropyl methyl cellulose), CMC (carboxymethyl cellulose), EHEC (ethyl hydroxyethyl cellulose) and HEC (hydroxyethyl cellulose), such as HPMC, or mixtures of any of these.

Suitably the active ingredient comprises at least one polyunsaturated fatty acid, such as at least one omega-3 polyunsaturated acid, such as at least EPA and/or DHA, for example in free fatty acid form. Suitable active ingredients also include soybean oil or oleic acid. Suitably the spray dried powder comprises about 70 wt % to about 90 wt % of the active ingredient.

Suitably the diluent or carrier comprises mannitol and microcrystalline cellulose in a ratio of 2:1.

Suitably the mixture of powder and diluent or carrier is used to manufacture a solid dosage form, such as a tablet, sachet, granulate or capsule, such as a tablet containing 20-60 wt % of the active ingredient.

Suitably the calcium chloride is present at a concentration of 2-5 mM in the emulsion.

Suitably the cellulose derivative(s) is present at a concentration of 2 to 4 wt % in the emulsion.

Suitably the cellulose nanocrystals are present at a concentration of 0.5 to 1 wt % in the emulsion.

### BRIEF DESCRIPTION OF THE DRAWINGS

FIG. 1: Yield (%) for the spray-drying process of eight batches with varying compositions

FIG. 2. Hardness vs punch separation for compacted spray-dried emulsion:excipient

FIG. 3. Relative thickness increase of the compacted spray-dried emulsion:excipient

FIG. 4. Particle size distribution for re-dispersed tablets.

FIG. 5: Punch compaction profile used during compaction experiments on the tablet compactor simulator.

### FORMATION OF THE EMULSION

Polymeric Cellulose Derivative

Generally, a suitable emulsion is formed according to the current disclosure by firstly dissolving at least one polymeric cellulose derivative (referred to herein as “cellulose derivative”, such as HPMC) in water. It will be understood that by “water” it is meant a substantially aqueous system where very small amounts of impurities (for example other water miscible solvents) may be present.

In some alternative aspects, alternative water-soluble polymers may be used instead of a polymeric cellulose derivative. Suitable alternative water soluble polymers include synthetic polymers as well as those derived from natural materials. One example of a suitable alternative water soluble polymer is polyvinyl alcohol, PVA. Suitable properties of such alternative polymers may be those described below for cellulose derivatives.

Various cellulose derivatives may suitably be used, for example HPMC, EHEC, CMC and HEC, or mixtures of any of these, however HPMC and EHEC have higher surface activity (lower surface tension) which is thought to aid the emulsion process.

Suitably the polymeric cellulose derivative has a surface tension in water of less than 60, such as less than 55 mN/m.

Exemplary values for surface tension of polymeric cellulose derivative solutions (in water) are shown below (pure water has a surface tension of approximately 72 mN/m):

| Polymer | Surface Tension (mN/m) |
|---|---|
| 0.3% CMC | 68.3 ± 0.1 |
| 0.3% HEC | 63.5 ± 0.1 |
| 0.3% EHEC | 50.3 ± 0.1 |
| 0.3% HPMC | 47.3 ± 0.3 |

HPMC may be additionally advantageous as it is available in lower viscosity grades; lower viscosity may be useful in order to counterbalance the viscosity-increasing effect of the CNCs. It will be appreciated that if the mixture is too viscous, it will not be effectively emulsified and/or spray dried.

As described in the Examples, suitably sufficient polymeric cellulose derivative is used such that the final emulsion contains between 2 and 4 wt % of polymeric cellulose derivative for emulsions containing about 20 wt % oil. It will be understood that some viscous polymers require dilution for efficient emulsion formation. In such cases, about 10 wt % oil and 2 wt % polymeric cellulose derivative may conveniently be used.

Cellulose Nanocrystals

Cellulose nanocrystal (CNC) suspension in water is then added to the polymer solution. CNCs are generally isolated by acid extraction of cellulose, during which process disordered amorphous regions of the cellulose chains are differentially dissolved, leaving behind the intervening areas of crystalline material which are generally a few nanometres wide and up to hundreds of nanometres long. For CNCs isolated from microcrystalline Cellulose (MCC) length and width ranges from 35-265 nm and 3-48 nm respectively, whereas crystals, for example, isolated from cotton have a length and width of 70-300 nm and 5-15 nm. The length of the CNC fibres may be measured by means of atomic force microscopy (AFM).

Such nanocrystals are commercially available, for example from CelluForce which markets CelluForce NCC™ derived from cellulose obtained from wood. These CNCs have a nominal average length of 150 nm and a nominal average diameter of 7.5 nm. As described in the Examples, suitably sufficient CNC is added that the emulsion contains 0.5-1 weight % (wt %) CNC.

Polyvalent Metal Ion Salt

Optionally, a pharmaceutically-acceptable salt with a polyvalent metal cation is added.

In one aspect, a pharmaceutically-acceptable salt with a polyvalent metal cation is added. In another aspect, a pharmaceutically-acceptable salt with a polyvalent metal cation is not added.

The term “pharmaceutically-acceptable salt” in this context means that the salt should generally be regarded (for example by the Regulatory bodies who authorise approvals of new medicines, such as the US Food and Drug Administration) as safe to use in medicines in humans in the quantities to be used in the compositions disclosed herein. This may restrict, for example, the metals which may be used.

The metal salt should be formed with a metal cation which has a valency of >1, that is, is “multivalent”.

Suitable metals for use in the salt include, but are not restricted to, calcium and magnesium.

The metal salt selected must also be sufficiently soluble that it can be dissolved to give a concentration of about 2 to 5 mM in the emulsion.

Suitable examples of such metal salts include calcium chloride, as illustrated in the Examples.

The salt may be added as an aqueous solution, for example calcium chloride may be added as a 0.1M aqueous solution.

The addition of calcium chloride increases the viscosity of the emulsion, it pre-flocculates the CNCs and provides the emulsion droplets with a connected structure after formulation. This provides homogeneity and stability of the emulsions prior to spray drying. At least 1 mM, such as between 1 and 2 mM, such as about 2 mM of calcium ion, such as 2-5 mM of calcium ion may suitably be used in the emulsion.

Active Pharmaceutical Ingredient (the API)

The API is then added as an oil phase on top of the water phase containing the other ingredients. In theory the process described herein could be applied to any API which is an oil at room temperature (for example between 15 and 35° C.). Particular examples of oil phase APIs are those rich in polyunsaturated fatty acids (PUFAs), often derived from natural sources.

The Examples herein include soybean oil (which is rich in PUFAs in triglyceride form, particularly linoleic acid (omega-6) and oleic acid (omega-9)) and oleic acid.

Suitably, the emulsion contains about 20 wt % of the oil, such as 19.5-20.5 wt %, such as 19-21 wt %, such as 18-22 wt %, such as 15-25 wt %. In other embodiments, suitably the emulsion contains about 10% of the oil, or even about 5% of the oil, particularly where a more dilute emulsion is required to reduce viscosity as discussed herein.

Oils rich in omega-3, often derived from fish, have been implicated as potential therapies for a wide variety of indications but are currently approved for treatment of hypertriglyceridemia. Examples include Lovaza™ (a mixture of PUFAs, particularly omega-3 PUFAs eicosapentaenoic acid (EPA; 20:5 n-3)) and docosahexaenoic acid (DHA; 22:6 n-3), in ethyl ester form), Vascepa™ (purified EPA in ethyl ester form) and Epanova™ (a mixture of PUFAs in free fatty acid form, with EPA, DHA and docosapentaenoic acid (DPA, 22:5 n-3) as the most abundant).

Suitably the API is an oil rich in PUFAs, particularly rich in omega-3 and/or omega-6 fatty acids. In one aspect, the API is an oil rich in omega-3 such as oil derived from fish oil. In one embodiment, the API is an oil rich in EPA and/or DHA.

In one aspect the API is the oil contains PUFAs in ethyl ester form. In one embodiment of this aspect, the API is the oil in Lovaza™. In another embodiment of this aspect, the API is the oil in Vascepa™.

In another aspect, the oil contains PUFAs in free fatty acid form. In one embodiment of this aspect, the API is the oil in Epanova™ (USAN omega-3 carboxylic acids). The oil composition used in Epanova™ is described and exemplified in United States Patent

U.S. Pat. No. 9,050,309 and related patents/applications, see for example Table 10 of WO2013/103902. Where omega-3 carboxylic acids is referred to in the Examples, it is to be understood to refer to the active ingredient in Epanova™

In a further embodiment the oil comprises:

EPA, in a weight percent amount of 50% to 60%;

DHA in a weight percent amount of 15% to 25%;

DPA in a weight percent amount of 1% to 8%;

wherein at least 90%, for example at least 95%, by weight of the PUFA in the composition is present in free fatty acid form.

In a further embodiment the oil comprises:

EPA, in a weight percent amount of 50% to 60%;

DHA in a weight percent amount of 17% to 23%;

DPA in a weight percent amount of 1% to 8%;

wherein at least 90%, for example at least 95%, by weight of the PUFA in the composition is present in free fatty acid form.

Formation of the Emulsion

The emulsion is formed by homogenisation for 3-5 minutes using a speed of 13000 rpm.

Preferably the shaft of the homogeniser is initially positioned at the oil/water interface. Higher speeds may be used depending on the choice of oil; friction may potentially degrade the oil layer.

Spray Drying Process

The emulsion may be spray dried using conventional apparatus, such as a mini-spray drier B-290 (Buchi). Feed rates of 5.5-7.5 ml/min may be used. Inlet temperatures of 114-120° C. and outlet temperatures of 75-84° C. may be used. Further detailed conditions may be found in the Examples hereinafter.

Excipients

Prior to compaction to form tablets, the spray dried emulsions may be mixed with one or more excipients, such as one or more diluent, carrier, binder or disintegrant. Use of the excipients improves powder flowability and helps stabilise the tablets against oil loss during compaction as illustrated in the examples.

Suitably, a mixture of mannitol and/or microcrystalline cellulose (MCC) may be used, such as a 1:2 blend of mannitol:MCC.

Suitably a mixture of spray dried emulsion and excipients are used such that the oil content of the mixture pre-compaction is 20-60%.

Compaction.

The powder may generally be compacted into tablets using conventional apparatus, although the Examples were carried out in a compaction simulator.

It will be appreciated that excessive force on the powder during compaction may cause unwanted release of the oil API.

The skilled person will be able to adapt the compaction process in order to ensure stability of any particular API. As shown in the examples, punch separation (that is, the minimum distance between the two halves of the punch which is compacting the tablet) needs to increase as the percentage of API loaded increases, for example from about 2.8 mm-3.0 mm for 50% load to 3.2 mm for 70% load in certain systems. This reflects increased softness of the powder due to the increased oil content.

Compaction rate (corresponding to time of contact between punch and solid) may also be varied to ensure minimum oil release and/or avoid lamination of the resulting tablet.

In some embodiments, contact time may suitably be less than 0.1 sec. In other embodiments contact time may suitably be >0.1 sec, such as 0.1-0.2 sec, such as about 0.5. In other embodiments, contact time may suitably be >0.5 sec, such as >1 second, such as >2 seconds, such as >3 seconds, such as 3 to 6 seconds, such as 4 to 6 seconds. It will be understood that such variation may be a consequence of the nature of the API and/or the excipients.

However the skilled person will understand that variation of oil, cellulose derivative and excipients will have an effect on the desirable compaction rate and punch separation which may be readily determined on a case by case basis.

Coating

In order to ensure stability of the tablets, they may conveniently be coated with one or more layers. Such coatings may provide physical stability and potentially chemical stability (for example by preventing contact of the API with water, air and/or light). Conventional coatings may be used and may be colourless, or include additives to give a coloured finish.

It will be understood that the Examples described herein have been carried out on laboratory scale. The skilled person will be able to adapt the processes described herein to be carried out on a larger scale.

In another aspect there is provided a solid pharmaceutical composition comprising

i) a powder comprising an active pharmaceutical ingredient which exists as an oil at least between 15° C. and 35° C., dispersed in a solid matrix, said solid matrix comprising cellulose nanocrystals, at least one cellulose derivative, a soluble calcium salt; and

ii) one or more pharmaceutically-acceptable excipients.

In another aspect there is provided a solid pharmaceutical composition comprising

i) a powder comprising an active pharmaceutical ingredient which exists as an oil at least between 15° C. and 35° C., dispersed in a solid matrix, said solid matrix comprising cellulose nanocrystals, at least one cellulose derivative, calcium chloride; and

ii) one or more pharmaceutically-acceptable excipients.

In another aspect there is provided a solid pharmaceutical composition comprising

i) a powder comprising an active pharmaceutical ingredient which exists as an oil at least between 15° C. and 35° C., dispersed in a solid matrix, said solid matrix comprising cellulose nanocrystals, HPMC, a soluble calcium salt; and

ii) one or more pharmaceutically-acceptable excipients.

In another aspect there is provided a solid pharmaceutical composition comprising

i) a powder comprising an active pharmaceutical ingredient which comprises at least one PUFA, dispersed in a solid matrix, said solid matrix comprising cellulose nanocrystals, at least one cellulose derivative, a soluble calcium salt; and

ii) one or more pharmaceutically-acceptable excipients.

In another aspect there is provided a solid pharmaceutical composition comprising

i) a powder comprising an active pharmaceutical ingredient which comprises at least one PUFA, dispersed in a solid matrix, said solid matrix comprising cellulose nanocrystals, HPMC, a soluble calcium salt; and

ii) one or more pharmaceutically-acceptable excipients.

In another aspect there is provided a solid pharmaceutical composition comprising

i) a powder comprising an active pharmaceutical ingredient which comprises at least one PUFA, dispersed in a solid matrix, said solid matrix comprising cellulose nanocrystals, HPMC and calcium chloride; and

ii) one or more pharmaceutically-acceptable excipients.

In another aspect there is provided a solid pharmaceutical composition comprising

i) a powder comprising an active pharmaceutical ingredient which comprises at least one PUFA, dispersed in a solid matrix, said solid matrix comprising cellulose nanocrystals, HPMC and calcium chloride; and

ii) mannitol and microcrystalline cellulose.

In another aspect there is provided a solid pharmaceutical composition comprising

i) a powder formed by spray-drying an emulsion, said emulsion comprising at least one cellulose derivative, water, cellulose nanocrystals, a pharmaceutically-acceptable soluble calcium salt and an active pharmaceutical ingredient which exists as an oil at least between 15° C. and 35° C.; and

ii) one or more pharmaceutically-acceptable excipients.

In another aspect there is provided a solid pharmaceutical composition comprising

i) a powder formed by spray-drying an emulsion, said emulsion comprising at least one cellulose derivative, water, cellulose nanocrystals, calcium chloride and an active pharmaceutical ingredient which exists as an oil at least between 15° C. and 35° C.; and

ii) one or more pharmaceutically-acceptable excipients.

In another aspect there is provided a solid pharmaceutical composition comprising

i) a powder formed by spray-drying an emulsion, said emulsion comprising HPMC, water, cellulose nanocrystals, calcium chloride and an active pharmaceutical ingredient which exists as an oil at least between 15° C. and 35° C.; and

ii) one or more pharmaceutically-acceptable excipients.

In another aspect there is provided a solid pharmaceutical composition comprising

i) a powder formed by spray-drying an emulsion, said emulsion comprising HPMC, water, cellulose nanocrystals, calcium chloride and an active pharmaceutical ingredient which exists as an oil at least between 15° C. and 35° C.; and

ii) mannitol and microcrystalline cellulose.

In another aspect there is provided a solid pharmaceutical composition comprising

i) a powder formed by spray-drying an emulsion, said emulsion comprising HPMC, water, cellulose nanocrystals, calcium chloride and an active pharmaceutical ingredient which comprises at least one PUFA; and

ii) one or more pharmaceutically-acceptable excipients.

In another aspect there is provided a solid pharmaceutical composition comprising

i) a powder formed by spray-drying an emulsion, said emulsion comprising HPMC, water, cellulose nanocrystals, calcium chloride and an active pharmaceutical ingredient which comprises at least one PUFA; and

ii) mannitol and microcrystalline cellulose.

In a further aspect there is provided a solid pharmaceutical composition comprising

i) a powder formed by spray-drying an emulsion which is formed by steps a to e:
a) dissolving HPMC in water;
b) dispersing cellulose nanocrystals in the resulting solution;
c) adding a pharmaceutically-acceptable soluble calcium salt;
d) adding an active pharmaceutical ingredient which is an oil between at least 15° C. and 35° C.; and
e) emulsifying the resulting mixture; and

ii) one or more pharmaceutically-acceptable excipients.

In a further aspect there is provided a solid pharmaceutical composition comprising

i) a powder formed by spray-drying an emulsion which is formed by steps a to e:
a) dissolving HPMC in water;
b) dispersing cellulose nanocrystals in the resulting solution;
c) adding calcium chloride;
d) adding an active pharmaceutical ingredient which is an oil between at least 15° C. and 35° C.; and
e) emulsifying the resulting mixture; and

ii) one or more pharmaceutically-acceptable excipients.

In one aspect the API comprises at least one PUFA and the cellulose derivative is HPMC.

In another aspect the API comprises at least one PUFA in free fatty acid form and the cellulose derivative is HPMC.

In another aspect, the API comprises omega-3 carboxylic acids and the cellulose derivative is HPMC.

In one aspect the solid pharmaceutical composition is compacted into a tablet dosage form.

In one embodiment the API comprises at least one PUFA, the cellulose derivative is HPMC and the spray-dried emulsion is mixed with mannitol and microcrystalline cellulose prior to compaction.

In another embodiment the API comprises at least one PUFA in free fatty acid form, the cellulose derivative is HPMC and the spray-dried emulsion is mixed with mannitol and microcrystalline cellulose prior to compaction.

In another embodiment, the API comprises omega-3 carboxylic acids, the cellulose derivative is HPMC and the spray-dried emulsion is mixed with mannitol and microcrystalline cellulose prior to compaction.

In one embodiment, the emulsion comprises 2-4 wt % of a polymeric cellulose derivative (for example HPMC), 0.5-1 wt % of CNC, and 18-22 wt % of an API which is an oil at room temperature.

In one embodiment, the emulsion comprises 2-4 wt % of a polymeric cellulose derivative (for example HPMC), 0.5-1 wt % of CNC, and about 20 wt % of an API which is an oil at room temperature.

In another embodiment, the emulsion comprises 2-4 wt % of a polymeric cellulose derivative (for example HPMC), 0.5-1 wt % of CNC, and 18-22 wt % of an API which is comprises at least one PUFA.

In another embodiment, the emulsion comprises 2-4 wt % of a polymeric cellulose derivative (for example HPMC), 0.5-1 wt % of CNC, and about 20 wt % of an API which is comprises at least one PUFA.

In another embodiment, the emulsion comprises 2-4 wt % of a polymeric cellulose derivative (for example HPMC), 0.5-1 wt % of CNC, and 18-22 wt % of soybean oil.

In another embodiment, the emulsion comprises 2-4 wt % of a polymeric cellulose derivative (for example HPMC), 0.5-1 wt % of CNC, and about 20 wt % of soybean oil. In another embodiment, the emulsion comprises 2-4 wt % of a polymeric cellulose derivative (for example HPMC), 0.5-1 wt % of CNC, and 18-22 wt % of oleic acid.

In another embodiment, the emulsion comprises 2-4 wt % of a polymeric cellulose derivative (for example HPMC), 0.5-1 wt % of CNC, and about 20 wt % of oleic acid.

In another embodiment, the emulsion comprises 2-4 wt % of a polymeric cellulose derivative (for example HPMC), 0.5-1 wt % of CNC, and 18-22 wt % of omega-3 carboxylic acids.

In another embodiment, the emulsion comprises 2-4 wt % of a polymeric cellulose derivative (for example HPMC), 0.5-1 wt % of CNC, and about 20 wt % of omega-3 carboxylic acids.

In one embodiment, the emulsion comprises 2-4 wt % of a polymeric cellulose derivative (for example HPMC), 0.5-1 wt % of CNC, 18-22 wt % of an API which is an oil at room temperature and further comprises 2-5 mM calcium chloride.

In one embodiment, the emulsion comprises 2-4 wt % of a polymeric cellulose derivative (for example HPMC), 0.5-1 wt % of CNC, about 20 wt % of an API which is an oil at room temperature and further comprises 2-5 mM calcium chloride.

In another embodiment, the emulsion comprises 2-4 wt % of a polymeric cellulose derivative (for example HPMC), 0.5-1 wt % of CNC, 18-22 wt % of an API which is comprises at least one PUFA and further comprises 2-5 mM calcium chloride.

In another embodiment, the emulsion comprises 2-4 wt % of a polymeric cellulose derivative (for example HPMC), 0.5-1 wt % of CNC, about 20 wt % of an API which is comprises at least one PUFA and further comprises 2-5 mM calcium chloride.

In another embodiment, the emulsion comprises 2-4 wt % of a polymeric cellulose derivative (for example HPMC), 0.5-1 wt % of CNC, 18-22 wt % of soybean oil and further comprises 2-5 mM calcium chloride.

In another embodiment, the emulsion comprises 2-4 wt % of a polymeric cellulose derivative (for example HPMC), 0.5-1 wt % of CNC, about 20 wt % of soybean oil and further comprises 2-5 mM calcium chloride.

In another embodiment, the emulsion comprises 2-4 wt % of a polymeric cellulose derivative (for example HPMC), 0.5-1 wt % of CNC, 18-22 wt % of oleic acid and further comprises 2-5 mM calcium chloride.

In another embodiment, the emulsion comprises 2-4 wt % of a polymeric cellulose derivative (for example HPMC), 0.5-1 wt % of CNC, about 20 wt % of oleic acid and further comprises 2-5 mM calcium chloride.

In another embodiment, the emulsion comprises 2-4 wt % of a polymeric cellulose derivative (for example HPMC), 0.5-1 wt % of CNC, 18-22 wt % of omega-3 carboxylic acids and further comprises 2-5 mM calcium chloride.

In another embodiment, the emulsion comprises 2-4 wt % of a polymeric cellulose derivative (for example HPMC), 0.5-1 wt % of CNC, about 20 wt % of omega-3 carboxylic acids and further comprises 2-5 mM calcium chloride.

Further aspects comprise the powder formed by spray drying any of the above embodiments and tablets formed by compaction of the powder formed by spray drying any of the above embodiments.

Therapeutic Uses

Solid dosage forms described herein may be useful for therapeutic treatment of humans.

In one aspect there is provided the a solid pharmaceutical composition for use as a medicament, said solid pharmaceutical composition comprising

i) a powder formed by spray-drying an emulsion, said emulsion comprising at least one cellulose derivative, water, cellulose nanocrystals, pharmaceutically-acceptable salt of a polyvalent metal cation and an active pharmaceutical ingredient which exists an oil at least between 15° C. and 35° C.; and

ii) one or more pharmaceutically-acceptable excipients.

In a further aspect there is provided a solid pharmaceutical composition for use as a medicament, said solid pharmaceutical composition comprising

i) a powder formed by spray-drying an emulsion which is formed by steps a to e:
a) dissolving at least one cellulose derivative in water;
b) dispersing cellulose nanocrystals in the resulting solution;
c) adding pharmaceutically-acceptable salt of a polyvalent metal cation;
d) adding an active pharmaceutical ingredient which is an oil between at least 15° C. and 35° C.; and
e) emulsifying the resulting mixture; and

ii) one or more pharmaceutically-acceptable excipients.

For example, where the API is a PUFA composition rich in omega-3 fatty acids, such as omega-3 carboxylic acids, the solid dosage forms may for example be useful for treatment of hypertriglyceridemia and/or mixed dyslipidemia.

In one aspect there is provided a solid pharmaceutical composition for use as a medicament for the treatment of hypertriglyceridemia in a subject with plasma triglyceride levels above about 500 mg/dL, said solid pharmaceutical composition comprising

i) a powder formed by spray-drying an emulsion, said emulsion comprising at least one cellulose derivative, water, cellulose nanocrystals, a pharmaceutically-acceptable salt of a polyvalent metal cation and an active pharmaceutical ingredient which exists an oil at least between 15° C. and 35° C.; and

ii) one or more pharmaceutically-acceptable excipients.

In a further aspect there is provided a solid pharmaceutical composition for use as a medicament for the treatment of hypertriglyceridemia in a subject with plasma triglyceride levels above about 500 mg/dL, said solid pharmaceutical composition comprising

i) a powder formed by spray-drying an emulsion which is formed by steps a to e:
a) dissolving at least one cellulose derivative in water;
b) dispersing cellulose nanocrystals in the resulting solution;
c) adding pharmaceutically-acceptable salt of a polyvalent metal cation;
d) adding an active pharmaceutical ingredient which is an oil between at least 15° C. and 35° C.; and
e) emulsifying the resulting mixture; and

ii) one or more pharmaceutically-acceptable excipients.

In one aspect there is provided a solid pharmaceutical composition for use as a medicament for the treatment of mixed dyslipidemia, said solid pharmaceutical composition comprising

i) a powder formed by spray-drying an emulsion, said emulsion comprising at least one cellulose derivative, water, cellulose nanocrystals, pharmaceutically-acceptable salt of a polyvalent metal cation and an active pharmaceutical ingredient which exists an oil at least between 15° C. and 35° C.; and

ii) one or more pharmaceutically-acceptable excipients.

In a further aspect there is provided a solid pharmaceutical composition for use as a medicament for the treatment of mixed dyslipidemia, said solid pharmaceutical composition comprising

i) a powder formed by spray-drying an emulsion which is formed by steps a to e:
a) dissolving at least one cellulose derivative in water;
b) dispersing cellulose nanocrystals in the resulting solution;
c) adding pharmaceutically-acceptable salt of a polyvalent metal cation;
d) adding an active pharmaceutical ingredient which is an oil between at least 15° C. and 35° C.; and
e) emulsifying the resulting mixture; and

ii) one or more pharmaceutically-acceptable excipients.

In one aspect there is provided a method of treating hypertriglyceridemia in a subject with plasma triglyceride levels above about 500 mg/dL comprising administering a solid pharmaceutical composition, said composition comprising:

i) a powder formed by spray-drying an emulsion, said emulsion comprising at least one cellulose derivative, water, cellulose nanocrystals, pharmaceutically-acceptable salt of a polyvalent metal cation and an active pharmaceutical ingredient which exists an oil at least between 15° C. and 35° C.; and

ii) one or more pharmaceutically-acceptable excipients.

In a further aspect there is provided a method of treating hypertriglyceridemia in a subject with plasma triglyceride levels above about 500 mg/dL comprising administering a solid pharmaceutical composition, said solid pharmaceutical composition comprising

i) a powder formed by spray-drying an emulsion which is formed by steps a to e:
a) dissolving at least one cellulose derivative in water;
b) dispersing cellulose nanocrystals in the resulting solution;
c) adding pharmaceutically-acceptable salt of a polyvalent metal cation;
d) adding an active pharmaceutical ingredient which is an oil between at least 15° C. and 35° C.; and
e) emulsifying the resulting mixture; and

ii) one or more pharmaceutically-acceptable excipients.

In one aspect there is provided a method of treating mixed dyslipidemia in a subject with plasma triglyceride levels above about 500 mg/dL comprising administering a solid pharmaceutical composition, said composition comprising

i) a powder formed by spray-drying an emulsion, said emulsion comprising at least one cellulose derivative, water, cellulose nanocrystals, pharmaceutically-acceptable salt of a polyvalent metal cation and an active pharmaceutical ingredient which exists an oil at least between 15° C. and 35° C.; and

ii) one or more pharmaceutically-acceptable excipients.

In a further aspect there is provided a method of treating mixed dyslipidemia in a subject with plasma triglyceride levels above about 500 mg/dL comprising administering a solid pharmaceutical composition, said solid pharmaceutical composition comprising

i) a powder formed by spray-drying an emulsion which is formed by steps a to e:
a) dissolving at least one cellulose derivative in water;
b) dispersing cellulose nanocrystals in the resulting solution;
c) adding pharmaceutically-acceptable salt of a polyvalent metal cation;
d) adding an active pharmaceutical ingredient which is an oil between at least 15° C. and 35° C.; and
e) emulsifying the resulting mixture; and

ii) one or more pharmaceutically-acceptable excipients.

Suitably the pharmaceutically-acceptable salt of a polyvalent metal cation is a soluble pharmaceutically-acceptable calcium salt, such as calcium chloride.

Suitably the cellulose derivative is HPMC.

Other suitable conditions and/or amounts of components have been described hereinbefore or are as illustrated in the Examples.

For the avoidance of doubt, although the emulsion could be spray dried onto inert (eg microcrystalline cellulose or sugar) cores, such embodiments are not preferred.

Fixed Dose Combinations

As described above, compositions disclosed herein may be useful either as mono-therapy or in combination with one or more additional active pharmaceutical ingredients.

Conveniently, such additional pharmaceutical ingredients are useful for treating cardiovascular diseases, in particular treatment of hyperlipidemia and/or hypertriglyceridemia.

In one aspect one or more additional active pharmaceutical ingredients are selected from lipid reducing agents, such as statins, fibrates/fibric acid derivatives.

In one aspect, a suitable additional active ingredient is a statin, conveniently selected from rosuvastatin, atorvastatin, simvastatin, fluvastatin, pravastatin and lovastatin.

In one aspect, an additional active ingredient, such as a statin, is mixed with the pharmaceutical composition disclosed herein, prior to compaction into a tablet or incorporation into a capsule. In another aspect an additional active ingredient, such as a statin, is spray coated onto the outside of a solid dosage form (such as a tablet or capsule) incorporating the pharmaceutical compositions disclosed herein.

### EXAMPLES

### Example 1—Emulsion Preparation

Preparation of Stock Solutions:

9 wt % aqueous stock solutions of hydroxypropyl methyl cellulose (HPMC, viscosity grade 6 cP and 50 cP, Shin-Etsu) were prepared by adding 27 g HPMC to 273 g water (Milli-Q, 18.2 MΩ) in a 500 mL glass vessel. The mixture was stirred at room temperature (magnetic stirrer, IKA-Kunkel) for at least 12 hours until all HPMC was dissolved.

4.8 wt % aqueous stock suspension of Cellulose Nanocrystal (CNC, Celluforce) was prepared by mixing 14.4 g CNC with 285.6 g water (Milli-Q, 18.2 MΩ) in a 500 mL glass bottle. The suspension was stirred at room temperature for at least 4 hours (magnetic stirrer, IKA-Kunkel) to ensure complete wetting of all CNC particles. The suspension was then sonicated using a sonication probe (Model CV334, Chemical Instruments AB) at 20% of maximum effect for 3×3 minutes, pause 1 minute.

0.1M aqueous stock solution of calcium chloride dihydrate was prepared by adding 1.46 g of calcium chloride dihydrate (Sigma) to 98.5 g water (Milli-Q, 18.2 MΩ) in a 200 mL glass bottle. The glass bottle was shaken by hand until all calcium chloride dihydrate was dissolved.

Preparation of Emulsion:

Amounts according to Table 1a of the stock solutions of HPMC, CNC and calcium chloride was added (in this order) to a 250 mL glass bottle. Additional water (Milli-Q, 18.2 MΩ) according to Table 1a was added to give 80 g aqueous phase. 20 g of either soybean oil (glycine max, Sigma) or oleic acid (general purpose grade, Fisher), was added on top of the aqueous phase. The formulation was homogenized for 3-5 minutes (diax 900 homogenizer, Heidolph Instruments) at 13000 rpm, with the shaft of the homogenizer initially positioned at the oil/water interface. 100 g of each emulsion was prepared.

The compositions of the emulsions prepared are shown in Table 1b.

TABLE 1a
Amount of stock solutions used to prepare 100 g emulsions.

| | HPMC | CNC | CaCl₂ | |
|---|---|---|---|---|
| | (9 wt %) | (4.8 wt %) | (0.1M) | Water |
| Sample | g | g | g | g |
| 4-1-O | 44.4 | 20.8 | 5.0 | 9.8 |
| 4-0.5-O | 44.4 | 10.4 | 5.0 | 20.2 |
| 2-1-O | 22.2 | 20.8 | 5.0 | 32.0 |
| 2-0.5-O | 22.2 | 10.4 | 5.0 | 42.4 |
| 4-1-S | 44.4 | 20.8 | 5.0 | 9.8 |
| 4-0.5-S | 44.4 | 10.4 | 5.0 | 20.2 |
| 4-0.5-S(50 cP)¹ | 44.4 | 10.4 | 5.0 | 20.2 |
| 2-1-S | 22.2 | 20.8 | 5.0 | 32.0 |
| 2-0.5-S | 22.2 | 10.4 | 5.0 | 42.4 |

¹Emulsion 4-0.5-S(50 cP) was prepared with HPMC (viscosity grade 50 cP). All other emulsions were prepared with HMPC (viscosity grade 6 cP)

The samples were named according to their composition as shown in Table 1b.

TABLE 1b
Compositions of emulsions prepared.

| Sample | HPMC | CNC | Type | Oil |
|---|---|---|---|---|
| name | weight % | weight % | of oil | weight % |
| 4-1-O | 4 | 1 | Oleic Acid | 20 |
| 4-0.5-O | 4 | 0.5 | Oleic Acid | 20 |
| 2-1-O | 2 | 1 | Oleic Acid | 20 |
| 2-0.5-O | 2 | 0.5 | Oleic Acid | 20 |
| 4-1-S | 4 | 1 | Soybean Oil | 20 |
| 4-0.5-S | 4 | 0.5 | Soybean Oil | 20 |
| 4-0.5-S(50 cP)² | 4 | 0.5 | Soybean Oil | 20 |
| 2-1-S | 2 | 1 | Soybean Oil | 20 |
| 2-0.5-5 | 2 | 0.5 | Soybean Oil | 20 |

²Emulsion 4-0.5-S(50 cP) was prepared with HPMC (viscosity grade 50 cP). All other emulsions were prepared with HMPC (viscosity grade 6 cP)

### Example 2—Spray-Drying of Emulsions

Emulsions 4-0.5-O, 2-1-O, 2-0.5-O, 4-0.5-S, 2-1-S and 2-0.5-S from Example 1 were spray-dried as produced. Emulsions 4-1-S and 4-1-0 were diluted 1.33 times (weight basis) with water (milli-Q, 18.2 MΩ) prior to spray drying. The emulsions were spray-dried (mini spray drier B-290, Buchi) at a feed rate of 5.5-7.5 ml/min using the two liquid nozzle and nitrogen as atomizing gas. Table 2 below describes the process conditions for the emulsions that were spray-dried. Powder was collected both from the collector and the cyclone.

TABLE 2
Process conditions for the nine spray-dried emulsions.

| | Mass | | | | | |
|---|---|---|---|---|---|---|
| | sprayed | Inlet T | Outlet T | Aspiration | Pump | |
| Sample | (g) | (° C.)² | (° C.)² | (%) | (%) | Q_{flow} |
| 4-1-S | 89.2 | 120-114 | 81-74 | 100 | 25 | 40-45 |
| 4-1-O | 90.7 | 114-114 | 79-75 | 100 | 25 | 40-45 |
| 4-0.5-S | 90.0 | 120-114 | 82-79 | 100 | 25 | 40-45 |
| 4-0.5-S(50 cP) | 90 | 90 | 50-60 | 100 | 10-15 | 40-45 |
| 4-0.5-O | 90.2 | 120-114 | 81-74 | 100 | 25 | 40-45 |
| 2-1-S | 83.8 | 120-114 | 82-76 | 100 | 25 | 40-45 |
| 2-1-O | 79.8 | 120-119 | 84-78 | 100 | 25 | 40-45 |
| 2-0.5-S | 84.4 | 120-114 | 82-76 | 100 | 25 | 40-45 |
| 2-0.5-O | 92.1 | 120-114 | 81-75 | 100 | 25 | 40-45 |

²Inlet and outlet temperatures varied between given intervals.
Q_{flow} = atomizing gas flow setting

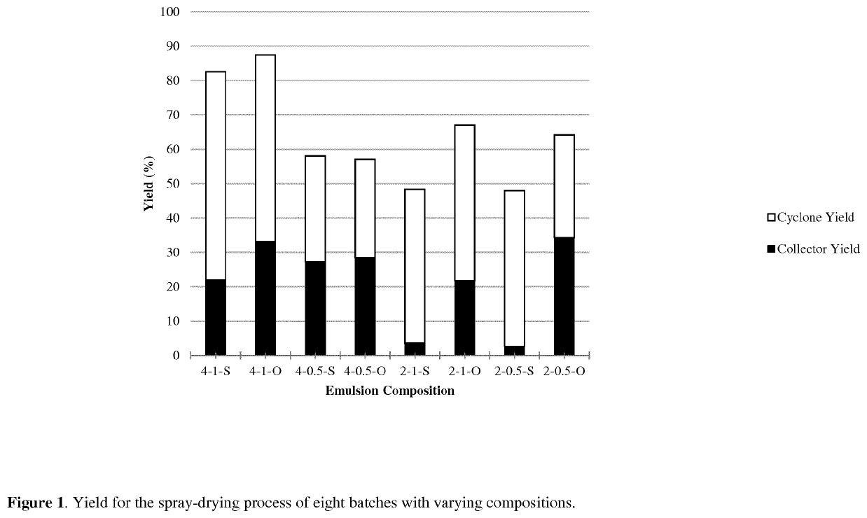

Emulsions 4-1-S and 4-1-O had a significantly improved yield in comparison with emulsions 4-0.5-S and 4-0.5-O, see FIG. 1. The yield for oleic acid based emulsions (4-1-O, 2-1-O and 2-0.5-O) were generally higher than its soybean oil counter-part (4-1-S, 2-1-S and 2-0.5-S), with the exception of emulsions 4-0.5-S and 4-0.5-O for which the yields were similar. There were no signs of phase separation in the above mentioned emulsions.

### Example 3—Fluidized Bed Drying of Emulsions

Microcrystalline Cellulose (MCC, PH102 batch 300017-01) cores were fluidized followed by the slow addition of emulsion 4-1-O (20% weight) in order to coat the cores (final product denoted batch 20% 4-1-O FB). This process was performed extremely slowly to avoid agglomeration of the particles, something which was observed at higher rates.

### Example 4—Compaction of Solid Emulsion Powder

Compaction of Spray-Dried Emulsions

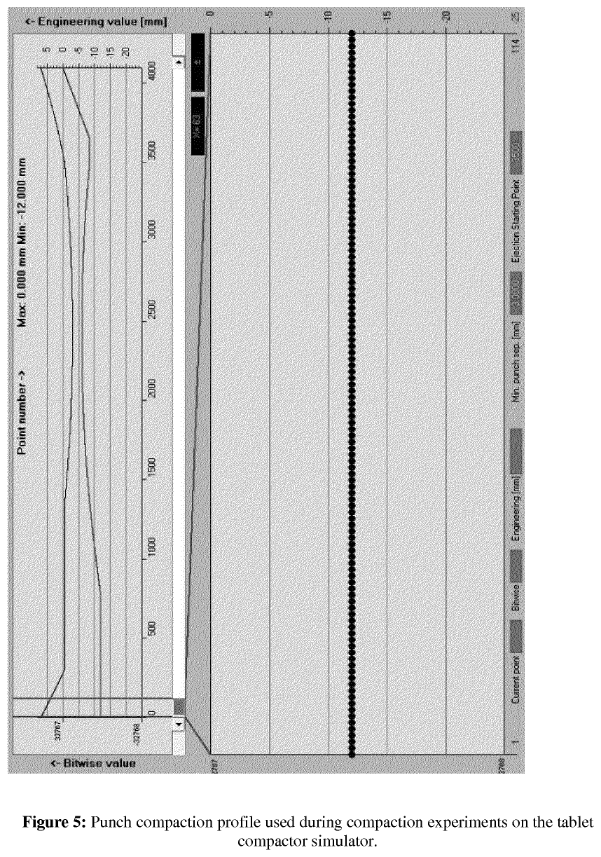

Compaction was performed with a tablet compactor simulator (ESH testing, Phoenix Services Ltd). The punches were flat-faced with a diameter of 10 mm. The punch compaction profile is shown in FIG. 5. All samples were prepared with a tablet mass of 300 mg. Spray-dried emulsions with the composition 4-0.5-S(50 cP) were initially compacted with variation in time and strain, in order to find suitable conditions. The spray-dried emulsion powders (from Example 2) were manually pre-compacted with a spatula prior to compaction to ensure all powder was below the surface of the die. Minimal punch separation distance, contact time and time settings tested on the spray dried emulsions are shown in Table 3.

TABLE 3
Sample composition and process conditions for
spray-dried emulsion compactions.

| | Minimal punch | Contact | Time |
|---|---|---|---|
| | separation distance | time | setting |
| Sample | (mm) | (s) | (s) |
| 4-0.5-S(50 cP) | 3.0 | 0.1 | 1 |
| 4-0.5-S(50 cP) | 3.0 | 33.3 | 300 |
| 4-0.5-S(50 cP) | 3.0 | 1.5 | 10 |
| 4-0.5-S(50 cP) | 3.0 | 3.9 | 25 |
| 4-0.5-S(50 cP) | 2.5 | 4.3 | 25 |
| 4-0.5-S(50 cP) | 3.5 | 3.1 | 25 |
| 4-1-S | 3.5 | 2.9 | 25 |
| 4-1-O | 3.5 | 2.8 | 25 |
| 4-0.5-S | 3.5 | 2.8 | 25 |
| 4-0.5-O | 3.5 | 2.8 | 25 |
| 2-1-S | 3.5 | 2.5 | 25 |
| 2-1-O | 3.5 | 2.6 | 25 |
| 2-0.5-S | 3.5 | 2.2 | 25 |
| 2-0.5-O | 3.5 | 2.7 | 25 |
| 4-1-O | 3.0 | 3.6 | 25 |
| 4-0.5-O | 3.0 | 3.8 | 25 |
| 4-1-S | 3.0 | 3.5 | 25 |

During the compaction of the spray-dried emulsions, it was observed that powder escaped through the punch die cavity, resulting in an incomplete compaction.

Compaction of Spray-Dried Emulsion:Excipient Blends

For these compactions a 2:1 blend of microcrystalline cellulose (PH102 batch 300017-01):Mannitol (Partech M200 batch M608919) was added to the powder with a loading of either 50% or 30% by weight. Compaction was performed with a tablet compactor simulator (ESH testing, Phoenix Services Ltd). The punches were flat-faced with a diameter of 10 mm. The punch compaction profile is shown in FIG. 5. All samples were prepared with a mass of 300 mg. Spray-dried emulsion:excipient blends were added to fill up the whole cavity. The minimal punch separation distance was optimized to give minimal amount of oil leakage and maximum compaction. The spray dried emulsion:excipient blend compositions along with the punch separation and contact time tested are shown in Table 4.

TABLE 4
Sample composition and process conditions for the
compactions involving solid emulsions: excipient mixtures.

| Denotation | Identity | Weight % | Weight % | | |
|---|---|---|---|---|---|
| of Spray- | of Spray- | of spray- | of 2:1 | | |
| dried | dried | dried | Microcrystalline | Punch | |
| emulsion: | emulsion | emulsion | cellulose: | sepa- | |
| excipient | sample in | sample in | Mannitol | ration | Contact |
| blend | blend | blend | in blend | (mm) | time (s) |
| 50% 4-1-O | 4-1-O | 50 | 50 | 3.0 | 4.54 |
| 50% 4-1-O | 4-1-O | 50 | 50 | 3.2 | 0.05 |
| 50% 4-1-O | 4-1-O | 50 | 50 | 3.0 | 0.05 |
| 50% 4-1-O | 4-1-O | 50 | 50 | 3.2 | 5.34 |
| 50% 4-1-O | 4-1-O | 50 | 50 | 3.2 | 5.49 |
| 50% 4-1-O | 4-1-O | 50 | 50 | 3.0 | 4.43 |
| 50% 4-1-O | 4-1-O | 50 | 50 | 2.9 | 3.49 |
| 50% 4-1-O | 4-1-O | 50 | 50 | 2.9 | 3.45 |
| 50% 4-1-O | 4-1-O | 50 | 50 | 3.1 | 4.37 |
| 50% 4-1-S | 4-1-S | 50 | 50 | 3.2 | 5.72 |
| 50% 4-1-S | 4-1-S | 50 | 50 | 3.2 | 5.73 |
| 50% 4-1-S | 4-1-S | 50 | 50 | 3.0 | 4.35 |
| 50% 4-1-S | 4-1-S | 50 | 50 | 3.0 | 4.55 |
| 50% 4-1-S | 4-1-S | 50 | 50 | 2.9 | 3.46 |
| 50% 4-1-S | 4-1-S | 50 | 50 | 2.9 | 3.34 |
| 50% 4-1-S | 4-1-S | 50 | 50 | 3.2 | 0.06 |
| 0% (Pure | — | — | 100% | 2.3 | 0.10 |
| MCC) | | | microcrystalline | | |
| | | | cellulose | | |
| 0% (Pure | — | — | 100% | 2.4 | 0.09 |
| MCC) | | | microcrystalline | | |
| | | | cellulose | | |
| 20% 4-1-O | 4-1-O | 20 | microcrystalline | 2.3 | 0.07 |
| FB⁴ | | | cellulose cores | | |
| 20% 4-1-O | 4-1-O | 20 | microcrystalline | 2.3 | 5.30 |
| FB⁴ | | | cellulose cores | | |
| 100% 4-1-O | 4-1-O | 100 | 0 | 3.0 | 7.36 |
| 100% 4-1-O | 4-1-O | 100 | 0 | 3.0 | 13.82 |
| 70% 4-1-O | 4-1-O | 70 | 30 | 3.2 | 2.69 |
| 70% 4-1-O | 4-1-O | 70 | 30 | 3.4 | 3.39 |

⁴Sample from Example 3 (microcrystalline cellulose cores that have been coated with 20 weight % of the 4-1-O emulsion).

Tablets prepared with spray-dried emulsion loadings of 50 and 70 weight % resulted in a successful compaction process without any powder escaping from the punch die. Table 5 shows the tablet dimensions and weight after compaction for the different spray-dried emulsion:excipient blends. For contact times equal or shorter than 60 ms (row 2, 3 and 17 in Table 4) the tablets based on soy bean oil (row 17) laminated after compaction but tablets based on oleic acid (row 2 and 3) did not.

TABLE 5
Results from compaction of spray-dried emulsion: excipient
blends. Presented data are punch separation, contact time
between the punches and the powder, thickness and diameter
of final tablet, hardness and final weight of the tablet.

| Denotation of | | | | | | |
|---|---|---|---|---|---|---|
| Spray-dried | Sepa- | Contact | Thick- | | Hard- | |
| emulsion: | ration | time | ness | Diameter | ness | Weight |
| excipient blend | (mm) | (s) | (mm) | (mm) | (N) | (mg) |
| 50% 4-1-O | 3.0 | 4.54 | 3.63 | 10.12 | 2.5 | 291.9 |
| 50% 4-1-O | 3.2 | 0.05 | 3.97 | 10.06 | 1.4 | 298.2 |
| 50% 4-1-O | 3.0 | 0.05 | 3.8 | 10.07 | 1.4 | 291 |
| 50% 4-1-O | 3.2 | 5.34 | 3.79 | 10.13 | 1.6 | 300.7 |
| 50% 4-1-O | 3.2 | 5.49 | 3.88 | 10.14 | 1.8 | 298 |
| 50% 4-1-O | 3.0 | 4.43 | 3.6 | 10.14 | 2.2 | 293.6 |
| 50% 4-1-O | 2.9 | 3.49 | 3.49 | 10.13 | 2.4 | 288.4 |
| 50% 4-1-O | 2.9 | 3.45 | 3.51 | 10.15 | 2.1 | 289.1 |
| 50% 4-1-O | 3.1 | 4.37 | 3.77 | 10.11 | | 295.5 |
| 50% 4-1-S | 3.2 | 5.72 | 3.62 | 10.1 | 3.6 | 297.5 |
| 50% 4-1-S | 3.2 | 5.73 | 3.6 | 10.08 | 3.6 | 297.8 |
| 50% 4-1-S | 3.0 | 4.35 | 3.43 | 10.08 | 5.5 | 292.4 |
| 50% 4-1-S | 3.0 | 4.55 | 3.35 | 10.09 | 4.7 | 290.9 |
| 50% 4-1-S | 2.9 | 3.46 | 3.26 | 10.11 | 5.5 | 282.9 |
| 50% 4-1-S | 2.9 | 3.34 | 3.23 | 10.11 | 5.4 | 285.1 |
| 50% 4-1-S | 3.2 | 0.06 | | | | |
| 0% (Pure MCC) | 2.3 | 0.10 | 2.7 | 10.07 | 3.4 | 299.9 |
| 0% (Pure MCC) | 2.4 | 0.09 | 3.01 | 10.07 | | 304.9 |
| 20% 4-1-O FB | 2.3 | 0.07 | | | | |
| 20% 4-1-O FB | 2.3 | 5.30 | | | | 281.9 |
| 100% 4-1-O | 3.0 | 7.36 | | | | |
| 100% 4-1-O | 3.0 | 13.82 | | | | |
| 70% 4-1-O | 3.2 | 2.69 | 3.87 | 9.98 | 1.1 | 289.5 |
| 70% 4-1-O | 3.4 | 3.39 | 4.05 | 10 | 1.2 | 298.7 |

A critical punch separation was identified to occur at around 2.9-3.0 mm for tablets produced with 50 weight % spray-dried emulsion:excipients and 3.2 mm for 70 weight % spray-dried emulsion:excipients. At the critical punch separation, a significant amount of oil separated from the tablet material, both visibly and gravimetrically. There were no issues with powder escaping the cavity with excipient addition. The compacted solid emulsion powder:excipient blends were visibly examined after compaction, and fully intact and compacted blends were obtained.

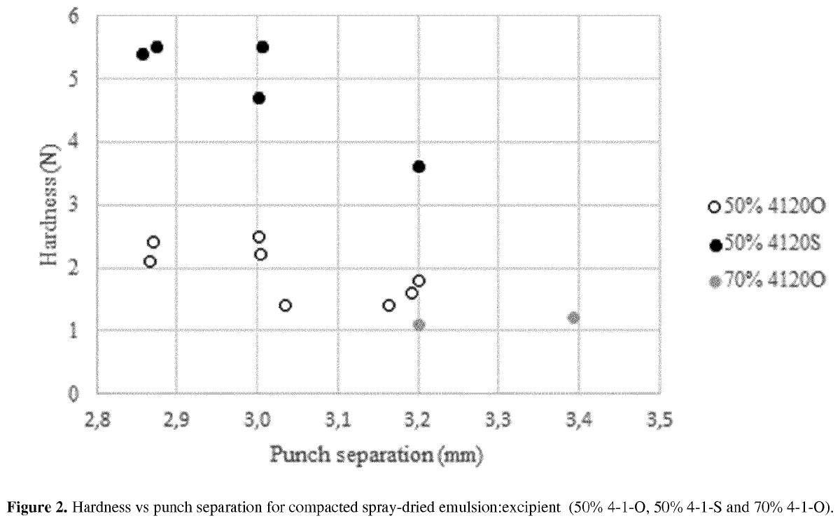

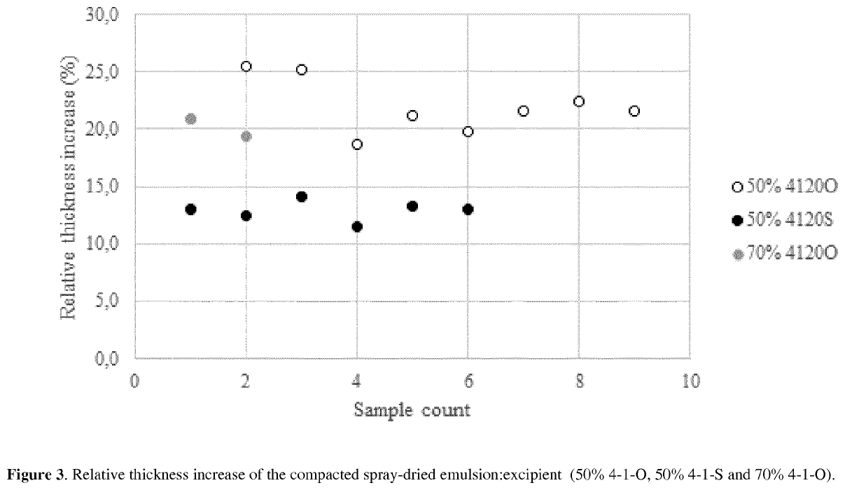

Tablet hardness was measured by a conventional tablet hardness tester (C50 tablet hardness tester, Holland) for some of the compacted formulations. The tablet's hardness versus punch separation, is shown in Table 5 and FIG. 2. FIG. 3 shows the relative thickness increase of the compacted tablets (comparing tablet thickness versus separation distance). The greater the increase in thickness, the greater the elasticity of the sample. FIG. 3 shows that the oleic acid based tablets have greater elasticity to the soybean tablets.

Compaction of emulsion coated microcrystalline cellulose cores (sample 20% 4-1-O FB) resulted in a poor material where neither compaction nor preservation of oil content was achieved.

### Example 5—Re-Dispersion of Spray-Dried Emulsion Powder and Tablets

A re-dispersion of the spray-dried emulsion powder was made by adding the spray-dried powder from Example 2 on top of water (Milli-Q) (final concentration of 20 mg/mL). The tablets from Example 4 were re-dispersed by adding the tablet (300 mg) into water (MilliQ, 15 mL). After a few hours, the vials were whisked gently in order to make the powder disperse homogenously in order to prepare the samples for light scattering.

Observations During Re-Dispersion

Tablets 50% 4-1-O, 50% 4-1-S and 70% 4-1-O (from Example 4) were re-dispersed in water.

In general, tablets containing 50% weight of spray-dried emulsion rapidly disintegrated following sinking of the tablet to the bottom of the vial after one minute, whilst tablets containing 70% weight of spray-dried emulsion floated in the vial for longer and had a different dispersive behaviour.

### Example 6—Sizing of the Emulsion Droplets in Freshly Prepared Emulsions, Emulsions from Re-Dispersed Spray-Dried Emulsions and Emulsions from Re-Dispersed Tablets

Sizing was performed with a Malvern Mastersizer 2000. Dispersion type for all measurements were liquid. Fresh emulsions were prepared 3-6 hours before analysis in order to look at the initial size of the droplets. The spray-dried emulsion powders from Example 2 were re-dispersed following the procedure in Example 5. The tablets from Example 4 were re-dispersed following the procedure in Example 5. Dispersions without excipients (for example, the spray-dried emulsions from Example 2 and the tablets described in Table 3) were stirred before sampling in order to get a more representative sizing. Microcrystalline cellulose containing samples (for example, tablets described in Table 4) were allowed to sediment for 10-20 minutes in order to avoid detecting a significant amount of microcrystalline cellulose particles. All samples were taken approximately in the middle of the liquid level in order to avoid withdrawing potentially phase separated oil from the top layer of the liquids.

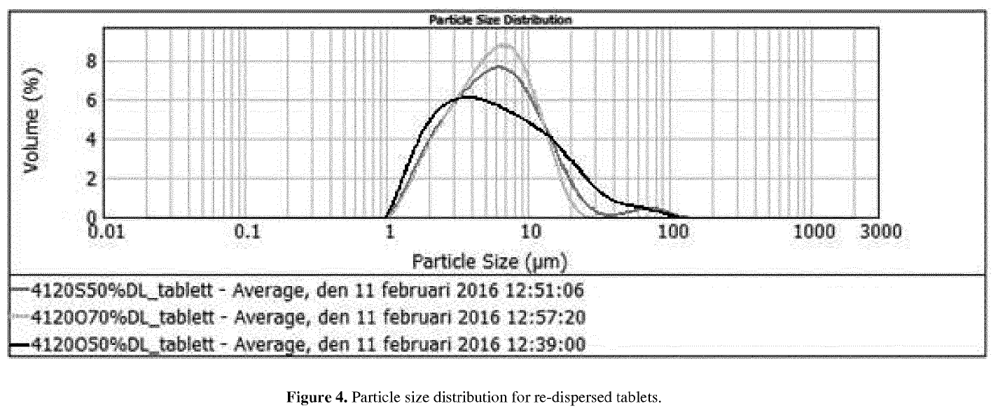

Table 6 shows the laser diffraction data for the samples tested, and FIG. 4 shows the laser diffraction data for three tablets (50% 4-1-S (table 4 row 18), 50% 4-1-O (Table 4 row 4) and 70% 4-1-O (Table 4 row 21) from Example 4)

TABLE 6
Laser diffraction data for the 4-1-emulsions, the data covers
newly produced emulsions (fresh), re-dispersed spray-dried
emulsions (SD emulsion) and re-dispersed tablets (tablet)

| Sample | d (0.1) [μm] | d (0.5) [μm] | d (0.9) [μm] |
|---|---|---|---|
| 4-1-S (fresh) | 2.67 | 6.03 | 11.40 |
| 4-1-S (SD | 2.23 | 5.39 | 11.55 |
| emulsions) | | | |
| 50% 4-1-S | 2.17 | 5.78 | 15.07 |
| (tablet) | | | |
| 4-1-O (fresh) | 1.57 | 2.22 | 3.28 |
| 4-1-O (SD | 1.87 | 3.95 | 10.63 |
| emulsions) | | | |
| 50% 4-1-O | 1.92 | 5.52 | 21.00 |
| (tablet) | | | |
| 70% 4-1-O | 2.22 | 5.66 | 12.26 |
| (tablet) | | | |

The difference between the laser diffraction data acquired for re-dispersed soybean spray-dried emulsion and fresh soybean emulsions was not large, indicating that the emulsion stability remained during spray-drying, assuming that the fresh emulsions were representative for the batch that was spray-dried.

The re-dispersed tablets exhibited a larger size distribution relative to their (prior to compaction) spray-dried emulsion form. It should be noted though that insoluble MCC-particles were present as excipient and may as such skew the size distribution. Regardless of potential skewing it can be seen that the size of droplets from the dispersed tablets were small enough to produce a stable emulsion system, and the compaction did not, in a catastrophic way, destroy the dispersed system.

Table 7 shows the laser diffraction results for fresh emulsion samples below where it can be seen that the cumulative majority of droplets across all samples are below 8 μm.

TABLE 7
Laser diffraction data for fresh emulsions, corresponding
to the same composition of those that were spray-dried.

| Sample | d (0.1) [μm] | d (0.5) μm] | d (0.9) μm] |
|---|---|---|---|
| 2-0.5-S | 1.99 | 4.85 | 13.04 |
| 2-1-S | 3.52 | 6.71 | 10.34 |
| 4-0.5-S | 3.40 | 7.70 | 13.67 |
| 4-1-S | 2.67 | 6.03 | 11.40 |
| 2-0.5-O | 1.87 | 3.68 | 7.85 |
| 2-1-O | 1.75 | 4.34 | 12.81 |
| 4-0.5-O | 1.64 | 3.04 | 5.69 |
| 4-1-O | 1.57 | 2.22 | 3.28 |

Storage over time of the emulsions (freshly prepared and/or re-dispersed emulsions from spray-dried emulsions or tablets prepared from spray-dried emulsions) may deteriorate over time with respect to droplet size.

### Example 7—Estimation of Oil Content in Spray-Dried Emulsions

0.25 g of spray-dried 4-0.5-S(50 cP) powder was mixed with hexane (20 mL) in order to leach encapsulated oil. The mixture was vigorously shaken for five minutes prior to centrifugation (thermo scientific heraeus labofuge 200, 4000 rpm, 15 minutes). The supernatant was carefully removed with a pipette, leaving the solids left in the vial. The process was repeated twice before leaving the solids to dry in an oven (100° C. for a few hours). The choice of hexane as leaching agent was based on the fact that HPMC is insoluble in hexane whilst soybean oil is soluble in hexane. The result was 82.5 wt % oil.

### Example 8 Omega 3-PUFA Emulsion Preparation and Tableting

Preparation of Stock Solutions:

9 wt % aqueous stock solutions of hydroxypropyl methyl cellulose (HPMC, viscosity grade 6 cP Shinetsu) were prepared by adding 27 g HPMC to 273 g water (Milli-Q, 18.2 MΩ) in a 500 mL glass vessel. The mixture was stirred at room temperature (magnetic stirrer, IKA-Kunkel) for at least 12 hours until all HPMC was dissolved.

4 wt % aqueous stock solutions of carboxymethyl cellulose (CMC, molecular weight 9000 g/mol, lot MKBT6160V, Sigma Aldrich) were prepared by adding 12 g CMC to 288 g water (Milli-Q, 18.2 MΩ) in a 500 mL glass vessel. The mixture was stirred at room temperature (magnetic stirrer, IKA-Kunkel) for at least 12 hours until all CMC was dissolved. 4 wt % aqueous stock solutions of hydroxyethyl cellulose (HEC, sample Lot#A-0028, Hercules' Aqualon) were prepared by adding 12 g HEC to 288 g water (Milli-Q, 18.2 MΩ) in a 500 mL glass vessel. The mixture was stirred at room temperature (magnetic stirrer, IKA-Kunkel) for at least 12 hours until all HEC was dissolved.

4.8 wt % aqueous stock suspension of Cellulose Nanocrystal (CNC, Celluforce) was prepared by mixing 14.4 g CNC with 285.6 g water (Milli-Q, 18.2 MΩ) in a 500 mL glass bottle. The suspension was stirred at room temperature for at least 4 hours (magnetic stirrer, IKA-Kunkel) to ensure complete wetting of all CNC particles. The suspension was then sonicated using a sonication probe (Model CV334, Chemical Instruments AB) at 20% of maximum effect for 3×3 minutes, pause 1 minute.

0.1M aqueous stock solution of calcium chloride dihydrate was prepared by adding 1.46 g of calcium chloride dihydrate (Sigma) to 98.5 g water (Milli-Q, 18.2 MΩ) in a 200 mL glass bottle. The glass bottle was shaken by hand until all calcium chloride dihydrate was dissolved.

Preparation of Emulsion:

Amounts according to Table 8a of the stock solutions of polymer, CNC and calcium chloride was added (in this order) to a 250 mL glass bottle. Additional water (Milli-Q, 18.2 MΩ) according to Table 8a was added to give the aqueous phase. Amounts of omega-3 carboxylic acids, Lot#38306, according to Table 8a, was added on top of the aqueous phase. The formulation was homogenized for 3-5 minutes (diax 900 homogenizer, Heidolph Instruments) at 13000 rpm, with the shaft of the homogenizer initially positioned at the oil/water interface. 150 g of each emulsion was prepared.

The compositions of the emulsions prepared are shown in Table 8b.

TABLE 8a
Amount of stock solutions, additional water and
omega-3 carboxylic acids (labelled as PUFA in the
Tables below) used to prepare 150 g emulsions.

| | | Polymer | | | | |
|---|---|---|---|---|---|---|
| | | stock | CNC | CaCl₂ | | |
| | | solution | (4.8 wt %) | (0.1M) | Water | PUFA |
| Sample | Polymer | g | g | g | g | g |
| 4-1-PUFA | HPMC | 66.67 | 31.25 | 7.5 | 44.58 | 30 |
| HPMC | | | | | | |
| 4-1-PUFA | HPMC | 66.67 | 31.25 | 0 | 52.08 | 30 |
| HPMC | | | | | | |
| no CaCl₂ | | | | | | |
| 4-1 PUFA | CMC | 75 | 15.63 | 3.75 | 40.62 | 15 |
| CMC | | | | | | |
| 4-1-PUFA | HEC | 37.5 | 7.81 | 1.88 | 95.31 | 7.5 |
| HEC | | | | | | |

TABLE 8b
Compositions of emulsions prepared using Omega-3 PUFA as oil

| | Polymer | Type | CNC | Oil | Water |
|---|---|---|---|---|---|
| | weight | of | weight | weight | weight |
| Sample | % | polymer | % | % | % |
| 4-1-PUFA HPMC | 4 | HPMC 6 cP | 1 | 20 | 75 |
| 4-1-PUFA HPMC | 4 | HPMC 6 cP | 1 | 20 | 75 |
| no CaCl₂ | | | | | |
| 4-1 PUFA CMC | 2 | CMC | 0.5 | 10 | 87.5 |
| 4-1-PUFA HEC | 1 | HEC | 0.25 | 5 | 93.75 |

Spray Drying of Omega-3 PUFA Emulsion:Excipient Blends

Emulsions from Table 8a and 8b were spray dried according to the procedure described in Example 2. The spray dried powders were used for subsequent compaction experiments.

Compaction of Spray-Dried Omega-3 PUFA Emulsion:Excipient Blends

Compaction experiments were performed on all spray dried emulsions in Table 8a and 8b. For these compactions a 2:1 blend of microcrystalline cellulose (PH102 batch 300017-01):Mannitol (Partech M200 batch M608919) was added to the powder with a loading of 50% by weight. Compaction was performed with a tablet compactor simulator (ESH testing, Phoenix Services Ltd). The punches were flat-faced with a diameter of 10 mm. The punch compaction profile is shown in FIG. 5. All samples were prepared with a mass of 300 mg. Spray-dried emulsion:excipient blends were added to fill up the whole cavity. The punch separation distance was set to 3.2 mm and profile duration time was set to 25 or 0.32 s. The final tablets were characterized by tablet weight, tablet thickness, tablet diameter, tablet hardness and disintegration according to European Pharmacopoeia methods. Data shown in Table 9.

TABLE 9
Results from compaction of spray-dried
Omega-3 PUFA emulsion: excipient blends

| Denotation of | | | | | Disinte- | Con- |
|---|---|---|---|---|---|---|
| Spray-dried | | Thick- | Diam- | Hard- | gration | tact |
| emulsion: | Weight | ness | eter | ness | time | time |
| excipient blend | (mg) | (mm) | (mm) | (N) | (s) | (s) |
| 4-1-PUFA | 291.5 | 3.90 | 10.14 | 0.8 | | 5.3 |
| HPMC^{a} | | | | | | |
| 4-1-PUFA | 294.4 | 3.87 | 10.14 | 1.2 | | 5.3 |
| HPMC^{a} | | | | | | |
| 4-1-PUFA | 292.6 | 3.93 | ND | <1 | | 5.3 |
| HPMC^{a} | | | | | | |
| 4-1-PUFA | 300.7 | 3.96 | 10.10 | 0.9 | | 0.07 |
| HPMC^{a} | | | | | | |
| 4-1-PUFA HPMC | 298.1 | 3.75 | 10.10 | 2.9 | | 0.07 |
| 4-1-PUFA HPMC | 308.4 | 3.85 | 10.14 | 2.8 | | 0.07 |
| 4-1-PUFA HPMC | 285.8 | 3.73 | 10.12 | 2.2 | | 0.07 |
| 4-1-PUFA HPMC | 275.3 | | | | | 0.07 |
| 4-1-PUFA HPMC | 280.7 | | | | 80 | 0.07 |
| 4-1-PUFA HPMC | 294.0 | | | | 75 | 0.07 |
| 4-1-PUFA HPMC | 294.3 | | | | 45 | 0.07 |
| 4-1-PUFA HPMC | 302.9 | | | | | 0.07 |
| 4-1-PUFA HPMC | 301.7 | | | | | 0.07 |
| 4-1-PUFA HPMC | 288.9 | 3.55 | 10.07 | 3.3 | | 0.07 |
| no CaCl₂ | | | | | | |
| 4-1-PUFA HPMC | 289.3 | 3.55 | 10.02 | 3.7 | | 0.07 |
| no CaCl₂ | | | | | | |
| 4-1-PUFA HPMC | 288.7 | 3.56 | 10.05 | 3.7 | | 0.07 |
| no CaCl₂ | | | | | | |
| 4-1-PUFA HPMC | 286.6 | | | | >3000^{c} | 0.07 |
| no CaCl₂ | | | | | | |
| 4-1-PUFA HPMC | 295.2 | | | | >3000^{c} | 0.07 |
| no CaCl₂ | | | | | | |
| 4-1-PUFA HPMC | 283.1 | | | | | 0.07 |
| no CaCl₂ | | | | | | |
| 4-1 PUFA CMC^{b} | | | | | | 0.07 |
| 4-1-PUFA HEC^{b} | | | | | | 0.07 |

^{a}Profile duration time: 25 s (all other were compressed with 0.32 s profile duration time to simulate typical large scale manufacturing process)
^{b}Not compressed into tablets
^{c}Tablet shape intact

### SUMMARY

Solid formulations (tablets) were successfully prepared and analysed from spray dried emulsions containing omega-3 carboxylic acids oil and excipients HPMC (with or without CaCl₂). Also, emulsions using CMC and HEC were prepared. Tablets with different composition exhibit different disintegration behavior.

1. A solid pharmaceutical composition comprising
i) a powder comprising an active pharmaceutical ingredient which exists an oil at least between 15° C. and 35° C., dispersed in a solid matrix, said solid matrix comprising cellulose nanocrystals, at least one cellulose derivative, optionally a pharmaceutically-acceptable salt of a polyvalent metal cation; and
ii) one or more pharmaceutically-acceptable excipients.

2. A solid pharmaceutical composition comprising
i) a powder formed by spray-drying an emulsion, said emulsion comprising at least one cellulose derivative, water, cellulose nanocrystals, an active pharmaceutical ingredient which exists an oil at least between 15° C. and 35° C., and optionally containing a pharmaceutically-acceptable salt of a polyvalent metal cation; and
ii) one or more pharmaceutically-acceptable excipients.

3. A solid pharmaceutical composition comprising
i) a powder formed by spray-drying an emulsion which is formed by steps a to d:
a) dissolving at least one cellulose derivative in water;
b) dispersing cellulose nanocrystals in the resulting solution;
c) optionally adding a pharmaceutically-acceptable salt of a polyvalent metal cation;
d) adding an active pharmaceutical ingredient which is an oil between at least 15° C. and 35° C.; and
e) emulsifying the resulting mixture; and
ii) one or more pharmaceutically-acceptable excipients.

4. A pharmaceutical composition according to claim 1 wherein the cellulose derivative is selected from HPMC, HEC, CMC and EHEC or mixtures of any of these.

5. A pharmaceutical composition according to claim 4 wherein the cellulose derivative is HPMC.

6. A pharmaceutical composition according to claim 1 wherein the pharmaceutically-acceptable salt of a polyvalent metal cation is a soluble pharmaceutically-acceptable calcium salt.

7. A pharmaceutical composition according to claim 1 wherein the pharmaceutically-acceptable salt of a polyvalent metal cation is calcium chloride.

8. A pharmaceutical composition according to claim 1 wherein the active ingredient comprises at least one polyunsaturated fatty acid.

9. A pharmaceutical composition according to claim 8 wherein the active ingredient comprises at least one omega-3 polyunsaturated acid.

10. A pharmaceutical composition according to claim 8 wherein the active ingredient comprises EPA and/or DHA.

11. A pharmaceutical composition according to claim 8 wherein the active ingredient comprises soybean oil or oleic acid.

12. A pharmaceutical composition according to claim 1 wherein the powder comprises about 70 wt % to about 90 wt % of the active ingredient.

13. A pharmaceutical composition according to claim 1 wherein the excipients comprise mannitol and microcrystalline cellulose in a ratio of 2:1.

14. A solid dosage form comprising the composition of claim 1.

15. A solid dosage form according to claim 14 which is a tablet.

16. A solid dosage form according to claim 14 containing 20-60 wt % of the active ingredient.

17. A composition according to claim 2 wherein the pharmaceutically-acceptable salt of a polyvalent metal cation is present at a concentration of 2-5 mM in the emulsion.

18. A composition according to claim 1 wherein the cellulose derivative is present at a concentration of 2 to 4 wt % in the emulsion.

19. A composition according to claim 1 wherein the cellulose nanocrystals are present at a concentration of 0.5 to 1 wt % in the emulsion.
